(19) United States
(12) Patent Application Publication
(10) Pub. No.: US 2024/0066265 A1
(43) Pub. Date: Feb. 29, 2024

(54) Catheter Assembly with Elongated Arm and Stabilization Platform

(71) Applicant: Becton, Dickinson and Company, Franklin Lakes, NJ (US)

(72) Inventors: Jonathan Karl Burkholz, Salt Lake City, UT (US); Serena Robinson Agrawal, Salt Lake City, UT (US); Jason Andrew Wine, Brea, CA (US); Dorien Gemma Butter, Millcreek, UT (US)

(21) Appl. No.: 18/238,592

(22) Filed: Aug. 28, 2023

Related U.S. Application Data
(60) Provisional application No. 63/401,925, filed on Aug. 29, 2022.

(51) Int. Cl. A61M 25/00

(52) U.S. Cl. CPC A61M 25/0097; A61M 2039/1077

(57) ABSTRACT

A catheter assembly includes a catheter adapter having a distal end, a proximal end, a lumen extending between the distal end and the proximal end, and a side port arranged between the distal end and the proximal end, with the side port in fluid communication with the lumen. The assembly further includes a catheter secured to the distal end of the catheter adapter and extending distally from the catheter adapter, a fluid conduit having a proximal end coupled to the side port and a distal end, with the fluid conduit in fluid communication with the side port, an elongated arm configured to extend distally away from the catheter adapter, and a stabilization platform spaced from the catheter adapter and positioned along the fluid conduit.

### CROSS-REFERENCE TO RELATED APPLICATION

The present application claims priority to U.S. Provisional Application No. 63/401,925 entitled “Catheter Assembly with Elongated Arm and Stabilization Platform” filed Aug. 29, 2022, the entire disclosure of which is hereby incorporated by reference in its entirety.

### BACKGROUND OF THE INVENTION

### Field of the Invention

The present disclosure relates to a catheter assembly with an elongated arm and stabilization platform.

### Description of Related Art

A vascular access device (VAD) may access peripheral vasculature of a patient. A VAD may be indwelling for short term (days), moderate term (weeks), or long term (months to years). VADs may be used for infusion therapy and/or for blood withdrawal.

A common type of VAD is an over-the-needle peripheral intravenous catheter (PIVC). Currently, there may be several limitations to the use of a PIVC for fluid infusion or blood draw. Among these limitations is the manipulation of the PIVC, for example, to manually connect various instruments thereto to enable blood draw; however, doing so in an uncontrolled way can increase incidence of dislodgement of the VAD from the vasculature, thus shortening the life of the catheter indwell. Accordingly, a need exists in the art for improved devices, systems, and methods of manipulating an indwelling catheter while preventing catheter dislodgement.

### SUMMARY OF THE INVENTION

In one aspect or embodiment, a catheter assembly includes a catheter adapter having a distal end, a proximal end, a lumen extending between the distal end and the proximal end, and a side port arranged between the distal end and the proximal end, with the side port in fluid communication with the lumen. The assembly further includes a catheter secured to the distal end of the catheter adapter and extending distally from the catheter adapter, a fluid conduit having a proximal end coupled to the side port and a distal end, with the fluid conduit in fluid communication with the side port, an elongated arm configured to extend distally away from the catheter adapter, and a stabilization platform spaced from the catheter adapter and positioned along the fluid conduit.

The assembly may further include a second elongated arm coupled to the catheter adapter and configured to extend distally away from the catheter adapter, where the elongated arms are arranged on opposite sides of the catheter adapter. The elongated arms may be configured to intersect one another at a location distal to the distal end of the catheter adapter. The stabilization platform may be coupled to at least one of the elongated arms. The stabilization platform may be arranged at an intersection of the elongated arms. The stabilization platform may be spaced from the elongated arms.

The fluid conduit may extend distally relative to the catheter. The assembly may further include an access connector arranged at the distal end of the fluid conduit. The access connector may include a needle-free connector. The stabilization platform may include an attachment member configured to secure the stabilization platform to a skin surface of a patient. The attachment member may include an adhesive pad.

The assembly may further include a clamp positioned along the fluid conduit, with the stabilization platform positioned between the clamp and the distal end of the fluid conduit. The assembly may further include a clamp positioned along the fluid conduit, with the stabilization platform positioned between the clamp and the side port of the catheter adapter. The stabilization platform may be moveable along the fluid conduit.

The assembly may further include a force controlled release connector coupled to the distal end of the fluid conduit, where at least a portion of the force controlled release connector is configured to separate from fluid conduit when a predetermined separation force is applied to the force controlled release connector. The force controlled release connector may include a disconnect connector and a release feature, with the release feature engaged with the disconnect connector and configured to be disengaged from the disconnect connector when a predetermined separation force is applied to the disconnect connector. The disconnect connector may include a valve member. The disconnect connector may include a first line extending from the disconnect connector and a second line extending from the disconnect connector, where the first line includes a first port and the second line includes a second port. The first and second ports may each include a needle-free connector.

### BRIEF DESCRIPTION OF THE DRAWINGS

FIG. 1 is a perspective view of a non-limiting embodiment of a prior art catheter assembly useful with blood draw devices as described herein;

FIG. 2 is a top view of a catheter assembly according to one aspect or embodiment of the present application;

FIG. 3 is a top view of a catheter assembly according to one aspect or embodiment of the present application;

FIGS. 4A-4B are top views of a catheter assembly according to one aspect or embodiment of the present application;

FIG. 5 is a top view of a catheter assembly according to one aspect or embodiment of the present application;

FIGS. 6A-6B are top views of a catheter assembly according to one aspect or embodiment of the present application;

FIG. 7 is a top view of a catheter assembly according to one aspect or embodiment of the present application;

FIG. 8 is a top view of a catheter assembly according to one aspect or embodiment of the present application;

FIG. 9 is a top view of a catheter assembly according to one aspect or embodiment of the present application;

FIG. 10 is a top view of a catheter assembly according to one aspect or embodiment of the present application;

FIG. 11 is a top view of a catheter assembly according to one aspect or embodiment of the present application;

FIG. 12 is a top view of a catheter assembly according to one aspect or embodiment of the present application;

FIG. 13 is a top view of a force controlled release connection according to one aspect or embodiment of the present application;

FIG. 14 is a cross-sectional view of a force controlled release connection according to a further aspect or embodiment of the present application; and

FIG. 15 is a top view of a force controlled release connection according to one aspect or embodiment of the present application.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, equivalents, variations, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

It should be understood that any numerical range recited herein is intended to include all values and sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value equal to or less than 10.

Provided herein are devices and systems for securing and/or stabilizing indwelling catheters, such as peripheral intravenous catheters (PIVCs). While certain devices (e.g., blood draw devices) are discussed below in terms of devices that may be used with PIVCs, as described herein, those of skill will appreciate that any number of different devices for introducing an instrument, including instruments ranging from tubes, probes, sensors, wiring, fiber optics, guidewires, etc., may be used within the scope of the present disclosure.

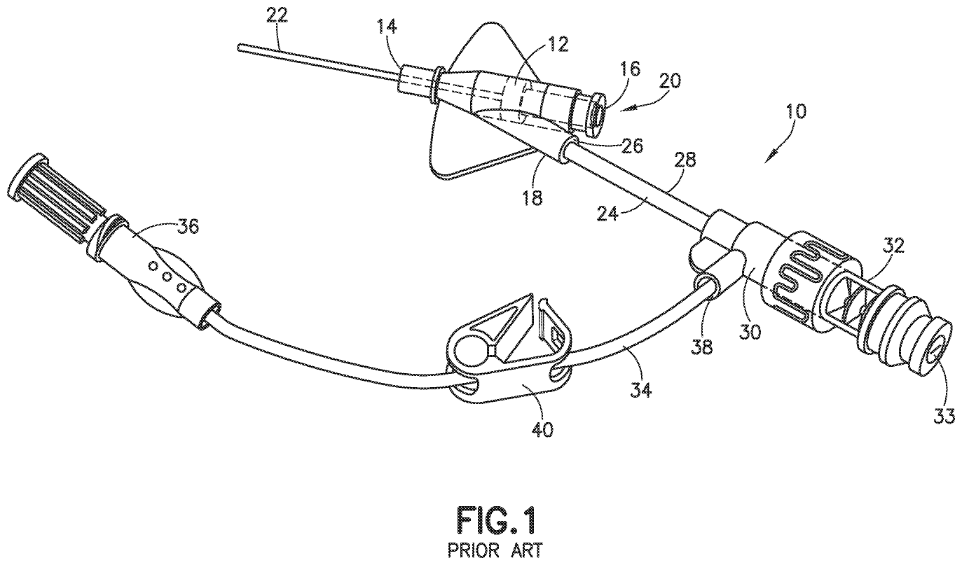

Referring now to FIG. 1, shown is a non-limiting embodiment of a prior art catheter assembly 10 useful with blood draw devices. Cather assembly 10 may include a catheter adapter 12, which may include a distal end 14 and a proximal end 16. In some embodiments, the catheter adapter 12 may include one or more additional ports 18. In some embodiments, port 18 may be disposed between the distal end 14 and the proximal end 16. In some embodiments, more than one port 18 may be disposed between the distal end 14 and the proximal end 16. In some embodiments, port 18 may be disposed at proximal end 16. In some embodiments, the first catheter adapter 12 may include a first lumen 20 extending through the distal end 14 and the proximal end 16. First lumen 20 may be sealed at proximal end 16 of catheter adapter 12.

In some non-limiting embodiments or aspects, the catheter assembly 10 may include a catheter 22 extending from the distal end 14. In some embodiments, the first catheter 22 may include a peripheral intravenous catheter, a midline catheter, or a peripherally-inserted central catheter. Catheter 22 may be formed of any suitable material and may be of any useful length, as known to those of skill in the art. In some non-limiting embodiments or aspects, the catheter assembly 10 may include a first fluid conduit 24 extending from the port 18. First fluid conduit 24 may be formed of any suitable material known to those of skill in the art, and may have a distal end 26 and a proximal end 28, and first fluid conduit 24 may be coupled, at distal end 26 thereof, to port 18. In some non-limiting embodiments or aspects, a connector 30 may be coupled to a proximal end 28 of first fluid conduit 24. Connector 30 may be a t-connector (e.g., one side port arranged at a 90 degree angle relative to a longitudinal axis of connector 30), a y-connector (e.g., one side port arranged at a 15-165 degree angle relative to a longitudinal axis of connector 30), or any other type of connector known in the art, and may include a second lumen therethrough, having any number of branches suitable for the type of connector.

In some non-limiting embodiments or aspects, catheter assembly 10 may include an extension set (integrated into or removably coupleable to catheter adapter 12, connector 30, and/or needleless access connector 32) including a second fluid conduit, such as second fluid conduit 34. Extension sets are known to those of skill in the art and are commercially available from, for example, Becton, Dickinson and Company. In some non-limiting embodiments or aspects, second fluid conduit 34 may include a luer connection 36 at an end thereof. In some non-limiting embodiments or aspects, the extension set may include a clamp 40, to allow for occlusion of second fluid conduit 34. Clamp 40 and second fluid conduit 34 may be formed of any suitable materials known to those of skill in the art. In non-limiting embodiments, second lumen (e.g., within connector 30) has an inner diameter that is substantially equivalent to an inner diameter of first fluid conduit 24 and/or second fluid conduit 34.

Catheter assembly 10 may include a needleless access connector 32 and/or a second fluid conduit 34. Suitable needleless access connectors 32 can include any split-septum connector and/or those with direct fluid path access. Needleless access connectors 32 are known to those of skill in the art and are commercially available from, for example, Becton, Dickinson and Company under the trade names MAXPLUS, MAXZERO, Q-SYTE, and SMARTSITE, and the NSYTE from NP Medical. While the non-limiting embodiments of FIG. 1 show needleless access connectors arranged at connector 30, those of skill in the art will appreciate that suitable needleless access connectors may also be arranged at luer connection 36. In non-limiting embodiments, needleless access connector 32 includes a septum 33, such as a self-healing septum. In non-limiting embodiments, the septum 33 is a slit-type septum. As will be described below, a blood draw device, as described herein, may be reversibly coupleable to the needleless access connector 32, and one or more portions of the blood draw device may pierce the septum 33.

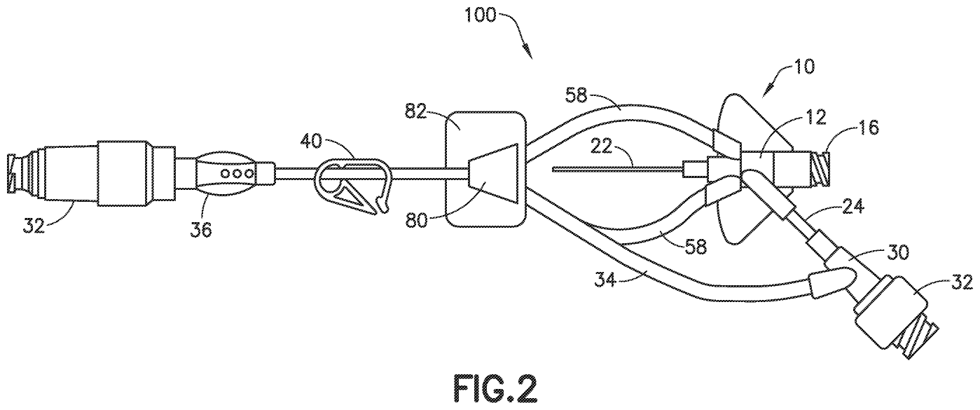

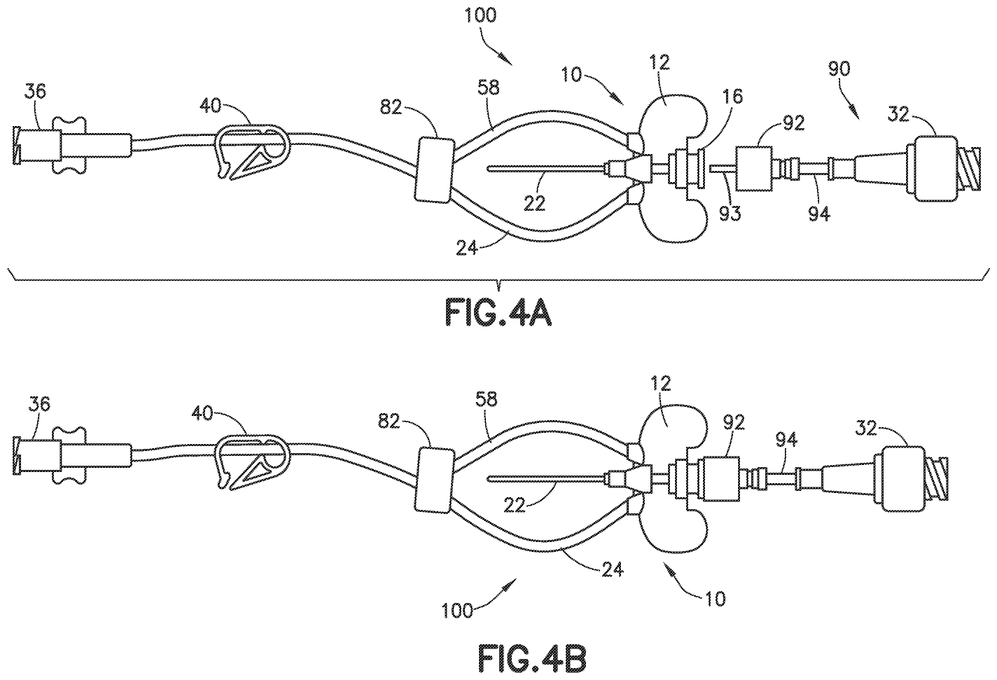

Turning to FIGS. 2-7, shown are non-limiting embodiments of a catheter assembly 100 including a catheter adapter 12 (optionally with a stabilization device), a second fluid conduit 34, and one or more elongated arms 58. One or more elongated arms 58 may be coupled to catheter adapter 12. Second fluid conduit 34 and/or one or more elongated arms 58 may be arranged on opposite sides of catheter adapter 12, and may be configured to extend distally (for example, as shown in FIGS. 4A-4B, 6A-6B, and 7) from catheter adapter 12. In non-limiting embodiments, second fluid conduit 34 and one or more elongated arms 58 extend distally along an arc, thereby intersecting distally of catheter adapter 12. At a point where second fluid conduit 34 and one or more elongated arms 58 intersect, a joint 80 and/or a stabilization platform 82 may be arranged, to couple second fluid conduit 34 and one or more elongated arms 58. Joint 80 and stabilization platform 82 may be arranged at different locations, for example, joint 80 may be arranged at an intersection of one or more elongated arms 58 and/or fluid conduits, and stabilization platform 82 may be arranged distally, for example, only coupled to a fluid conduit. Second fluid conduit 34 may include, at a distal end thereof, a connector, such as a luer connecter 36 and/or a needle-free connector (NFC) (also known as a needless access connector, NAC) 32. Those of skill will appreciate that NFCs/NACs 32, as described herein, may be integrated into conduits and/or connectors, or may be removably coupled to such structures. Catheter assembly 100 may further include a clamp 40, arrangeable anywhere along the length of second fluid conduit 34.

Catheter assembly 100 may be coupled to an instrument delivery device or other component through proximal end 16. As shown in FIGS. 4A-4B, catheter assembly 100 may be coupled to a further port, by coupling proximal end 16 of catheter adapter 12 to luer adapter 92. Luer adapter 92 may be coupled to a fluid conduit 94, which may have at an end thereof a NFC/NAC 32.

One or more elongated arms 58 may be formed of any suitable material. While not wishing to be bound by the theory, elongated arm(s) 58 with a material having a substantially similar elasticity to materials typically used in fluid conduits, such as second fluid conduit 34, may be preferable to minimize twisting of catheter adapter 12 when a distally-directed force is applied to one or more elongated arms 58 and/or second fluid conduit 34. By arranging one or more elongated arms 58 and/or second fluid conduit 34 in the manner shown in the accompanying figures, force can be applied to catheter adapter 12 and, consequently, catheter 22, whereby catheter 22 is pulled further into the vasculature, rather than inadvertently being pulled out of the vasculature if, for example, second fluid conduit 34 extended proximally.

The non-limiting embodiments of FIGS. 2-7 show various possible configurations of catheter assembly 100, but those of skill will appreciate that these should not be considered limiting. For example, in non-limiting embodiments, elongated arm(s) 58 may also be fluid conduits, coupled to one or more side ports 18 of catheter adapter 12. In non-limiting embodiments, catheter assembly 100 includes one or two elongated arms 58, one or more second fluid conduits 34, and various combinations thereof.

With continuing reference to the accompanying figures, stabilization platform 82 may be configured to be reversibly coupleable to the patient. For example, stabilization platform 82 may include a backing on a skin-facing surface that includes an adhesive. Suitable adhesives for reversibly coupling components to a patient's skin are known to those of skill in the art. Stabilization platform 82 may be formed of any suitable material, for example, flexible materials, soft materials, semi-rigid materials, or rigid materials. Stabilization platform 82 may also include one or more visible indicia, conveying information to healthcare professionals, such as catheter gauge, length, and/or information relating to one or more procedures to be performed through catheter assembly 100.

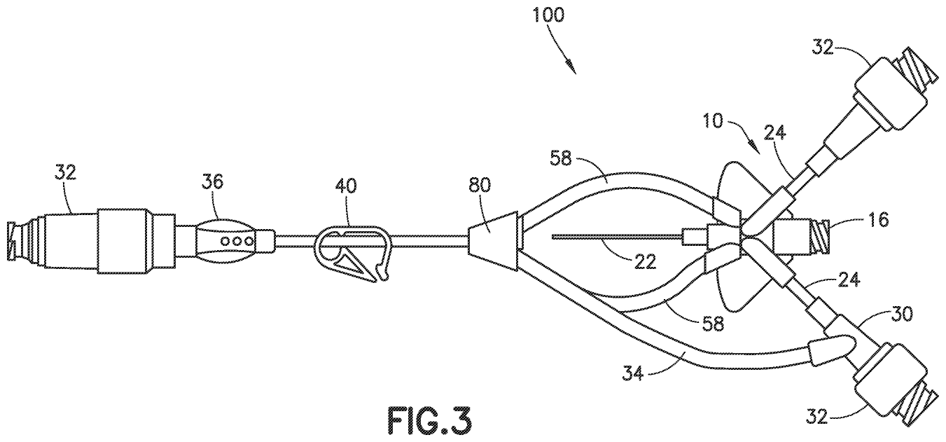

Turning to FIGS. 2 and 3, shown are non-limiting embodiments of catheter assembly 100, in which a plurality of elongated arms 58 are coupled to catheter adapter 12 and extend distally therefrom, and one (FIG. 2) or two (FIG. 3) proximally-directed side ports 18 are included. In both figures, a first fluid conduit 24 is coupled to one of the side ports, and has, at a distal end thereof, a connector 30 (which may include a NFC/NAC 32), the connector including a side port to which a second fluid conduit 34 is coupled. Second fluid conduit 34 is distally extending, and may, as shown in these non-limiting examples, intersect with elongated arms 58 at joint 80, which is arranged with stabilization platform 82. In the illustrated non-limiting embodiments, a clamp 40 is included on second fluid conduit 34, and at distal end of second fluid conduit 34 a luer connection 36 with a NFC/NAC 32 is arranged. In the non-limiting embodiment of FIG. 3, catheter adapter 12 includes a plurality of side ports 18, and a further first fluid conduit 24 is coupled to another side port. This further first fluid conduit 24 includes, at a distal end thereof, a NFC/NAC 32. Those of skill will appreciate that luer connection 36 and/or NFCs/NACs 32 in the embodiments of FIGS. 2 and 3, as well as any other embodiment discussed herein, may be replaced with any other suitable structure and/or connector.

Turning to FIGS. 4A-4B, shown are non-limiting embodiments of catheter assembly 100, prior to (FIG. 4A) and after (FIG. 4B) coupling of a near-patient access port (here, a NFC/NAC 32). The near-patient access port includes a luer adapter 92 for coupling to catheter adapter 12, a cannula 93, and a fluid conduit 94. In the illustrated non-limiting embodiment, catheter assembly 100 includes only a single elongated arm 58, and first fluid conduit 24 extends distally from side port 18, intersecting with elongated arm 58 at stabilization platform 82. In the illustrated non-limiting embodiments, a clamp 40 is included on first fluid conduit 24, and at distal end of first fluid conduit 24 a luer connection 36 is arranged.

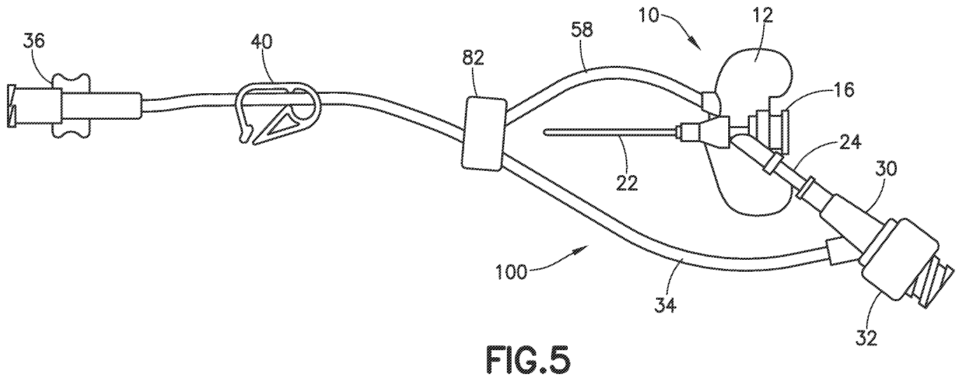

Turning to FIG. 5, shown is a non-limiting embodiment of catheter assembly 100 in which a single elongated arm 58 is coupled to catheter adapter 12 and extends distally therefrom, and one proximally-directed side port 18 is included. First fluid conduit 24 is coupled to the side port 18, and has, at a distal end thereof, a connector 30 (which may include a NFC/NAC 32), the connector including a side port to which second fluid conduit 34 is coupled. Second fluid conduit 34 is distally extending, and may, as shown in these non-limiting examples, intersect with elongated arm 58 at stabilization platform 82. In the illustrated non-limiting embodiments, a clamp 40 is included on second fluid conduit 34, and at distal end of second fluid conduit 34 a luer connection 36 is arranged.

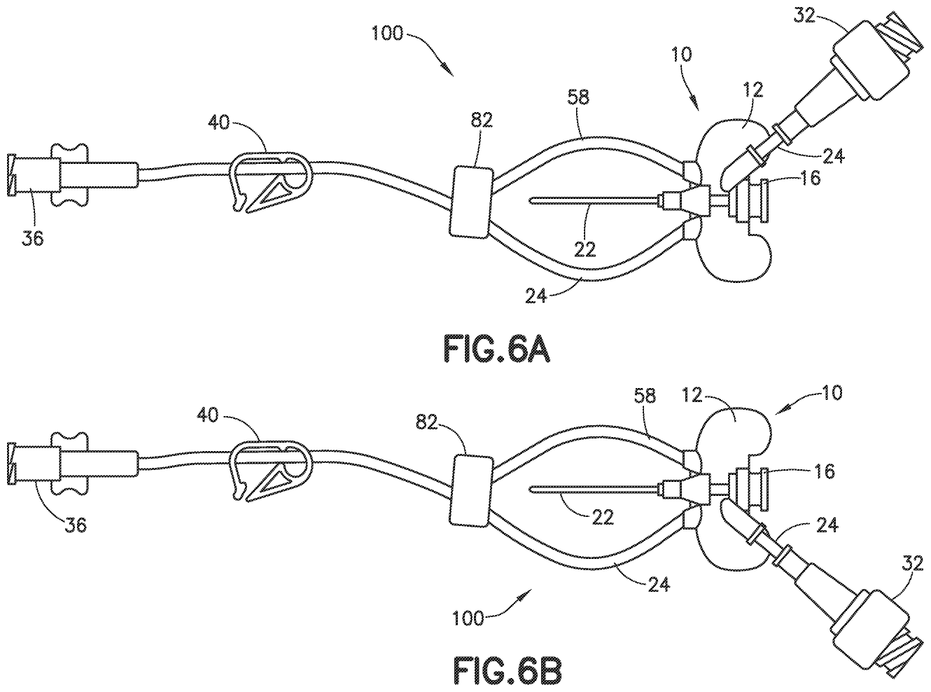

Turning to FIGS. 6A-6B, shown are non-limiting embodiments of catheter assembly 100. In the illustrated non-limiting embodiments, catheter assembly 100 includes only a single elongated arm 58, and first fluid conduit 24 extends distally from side port 18, intersecting with elongated arm 58 at stabilization platform 82. In the illustrated non-limiting embodiments, a clamp 40 is included on first fluid conduit 24, and at distal end of first fluid conduit 24 a luer connection 36 is arranged. In the illustrated non-limiting embodiments, catheter adapter 12 further includes a proximally-directed side port (on the opposite side (FIG. 5A) or the same side (FIG. 5B)), as the distally-directed side port to which first fluid conduit 24 is coupled), to which a further first fluid conduit 24 is coupled. This further first conduit 24 includes, at a distal end thereof, a NFC/NAC 32.

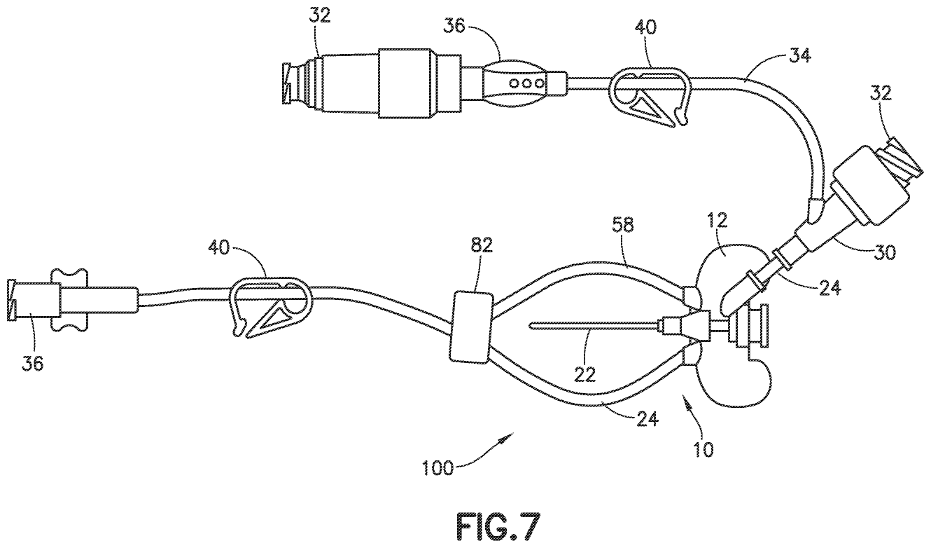

Turning to FIG. 7, shown is a non-limiting embodiment of catheter assembly 100. In the illustrated non-limiting embodiment, catheter assembly 100 includes only a single elongated arm 58, and first fluid conduit 24 extends distally from side port 18, intersecting with elongated arm 58 at stabilization platform 82. In the illustrated non-limiting embodiment, a clamp 40 is included on first fluid conduit 24, and at distal end of first fluid conduit 24 a luer connection 36 is arranged. In the illustrated non-limiting embodiment, catheter adapter 12 further includes a proximally-directed side port (on the opposite side, though those of skill will appreciate that other arrangements are possible, as the distally-directed side port to which first fluid conduit 24 is coupled), to which a further first fluid conduit 24 is coupled. This further first fluid conduit 24 includes, at a distal end thereof, a connector 30, which includes, as an integral or removable component, a NFC/NAC 32. Connector 30 includes a side port to which a second fluid conduit 34 is coupled. In the illustrated non-limiting embodiment, a clamp 40 is included on second fluid conduit 34, and at distal end of second fluid conduit 34 a luer connection 36 with a NFC/NAC 32 is arranged.

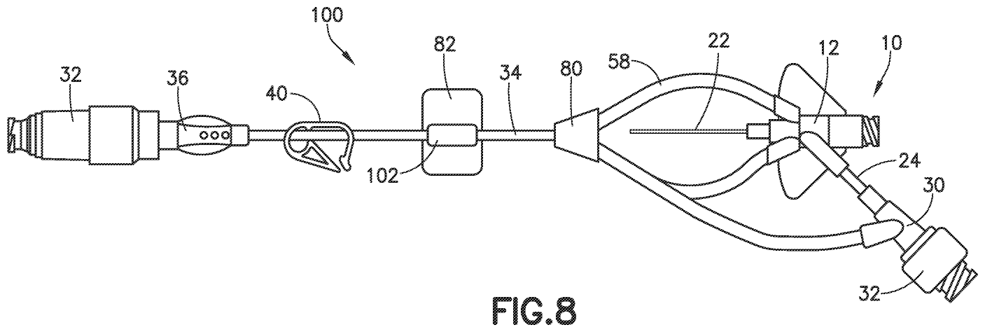

Referring to FIG. 8, shown is a non-limiting embodiment of catheter assembly 100. In the illustrated non-limiting embodiment, the stabilization platform 82 is spaced from the catheter adapter 12 and positioned along the first fluid conduit 24. The stabilization platform 82 is rectangular and includes a protrusion 102 that receives the first fluid conduit 24. The stabilization platform 82 may be moveable along the first fluid conduit 24 or fixed to the first fluid conduit 24.

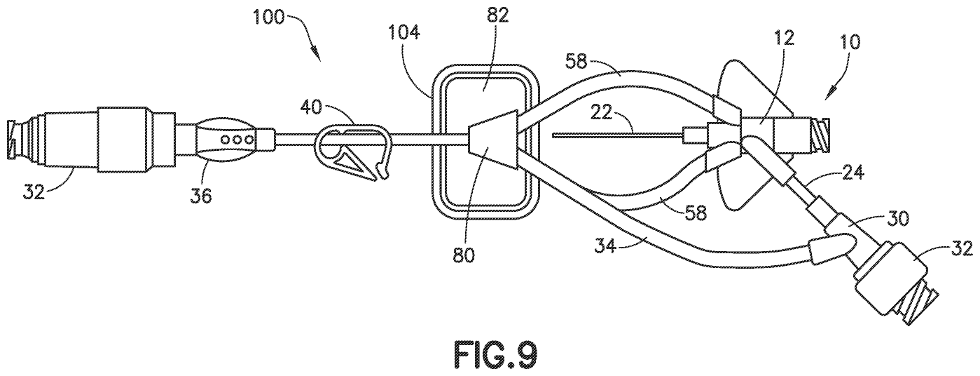

Referring to FIG. 9, shown is a non-limiting embodiment of catheter assembly 100. In the illustrated non-limiting embodiment, the stabilization platform 82 includes an attachment member 104 configured to secure the stabilization platform 82 to a skin surface of a patient. The attachment member 104 may include an adhesive pad. The attachment member 104 may be securement tape, an external stabilization device (ESD), dressing, and/or an anchoring platform adhesive with paper backing and skin adhesive. In one aspect or embodiment, the stabilization platform 82 is shaped to fit within the StatLock™ ESD product from Becton, Dickinson and Co. The stabilization platform 82 may be positioned between the clamp 40 and the distal end of the first fluid conduit 24 or may be positioned between the clamp 40 and the side port 18 of the catheter adapter 12.

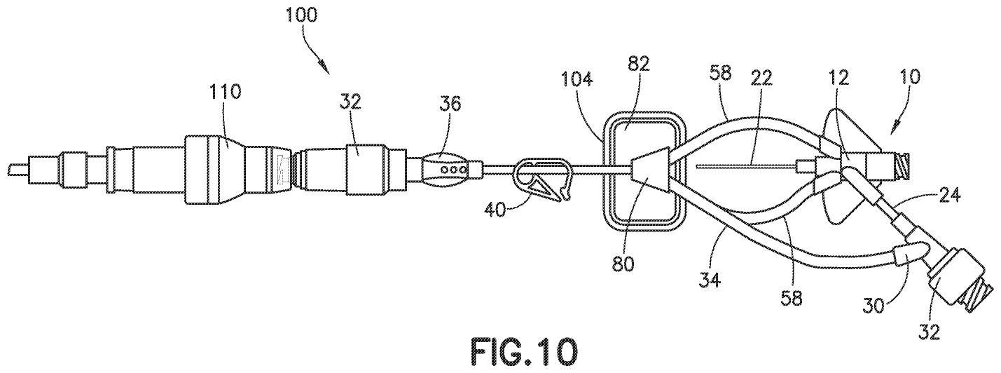

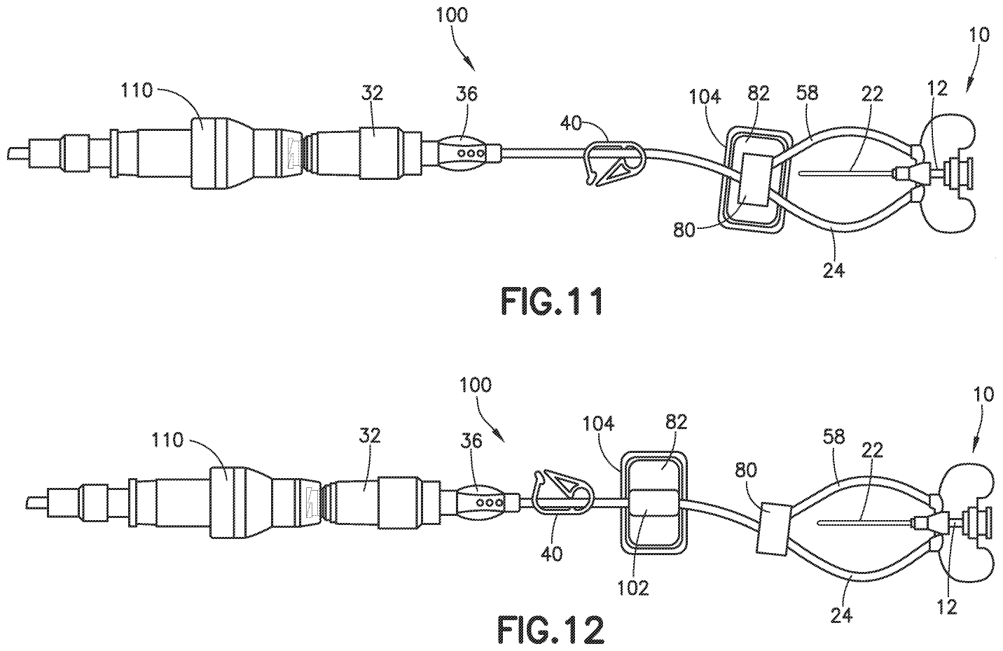

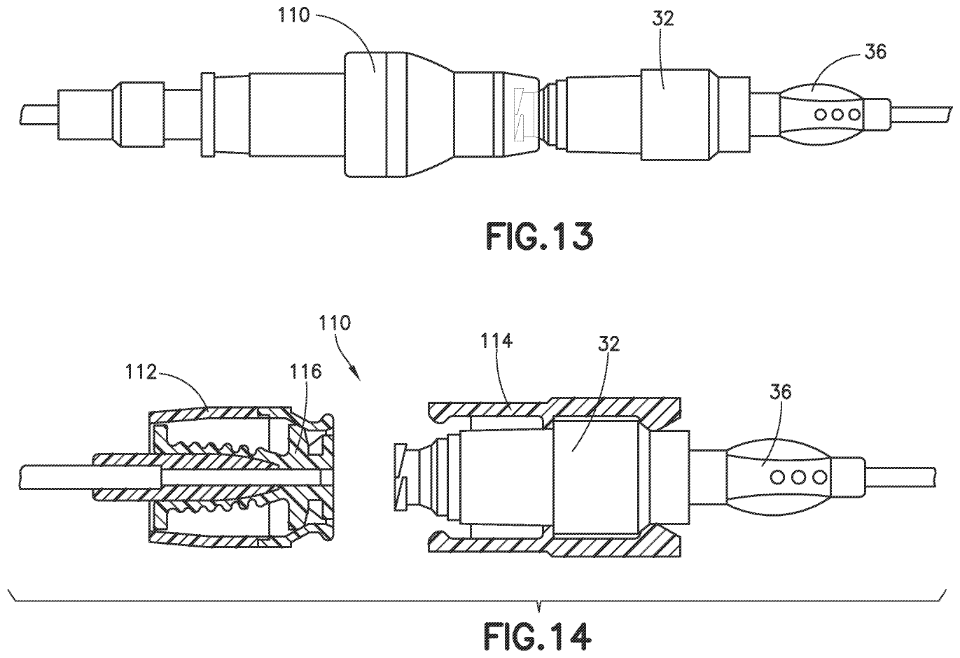

Referring to FIGS. 10-15, shown are non-limiting embodiments of catheter assembly 100. In the illustrated non-limiting embodiments, any of the embodiments of catheter assembly 100 discussed above may include a force controlled release connector 110 coupled to the distal end of the fluid conduit 24, 34. At least a portion of the force controlled release connector 110 is configured to separate from fluid conduit 24, 34 when a predetermined separation force is applied to the force controlled release connector 110. At least a portion of the force controlled release connector 110 is configured to detach from the fluid conduit 24, 34 to prevent excessive forces from being transmitted to the catheter adapter 12 via the fluid conduit 24, 34. For example, an intravenous line may be connected to the fluid conduit 24, 34 via the force controlled release connector 110 such that excessive movement of the intravenous line will merely detach the connection between the fluid conduit 24, 34 and the force controlled release connector 110 rather than disrupting the placement of the catheter 22.

Referring to FIG. 14, the force controlled release connector 110 includes a disconnect connector 112 and a release feature 114 coupled to the connector 30, 32, 36. The release feature 114 is engaged with the disconnect connector 112 and configured to be disengaged from the disconnect connector 112 when a predetermined separation force is applied to the disconnect connector 112 or the connector 30, 32, 36. The disconnect connector 112 includes a valve member 116, which is configured to seal the disconnect connector 112 when separated from the connector 30, 32, 36. In one aspect or embodiment, the release feature 114 is configured to be biased radially outward when the predetermined separate force is reached. The release feature 114 and the disconnect connector 112 may form a camming arrangement where an axial force causes the release feature 114 to move radially outward, thereby releasing the disconnect connector 112 from the release feature 114. The release feature 114 may be formed integrally with the connector 30 or may be a separate component attached or coupled to the connector 30.

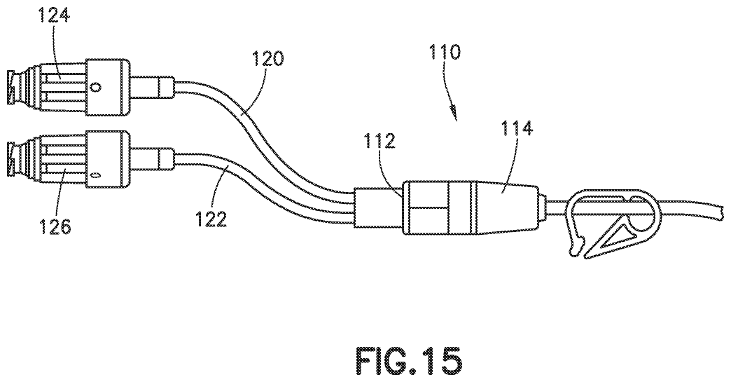

Referring to FIG. 15, in some aspects or embodiments, the disconnect connector 112 includes a first line 120 extending from the disconnect connector 112 and a second line 122 extending from the disconnect connector 112. The first line 120 includes a first port 124 and the second line 122 includes a second port 126. The first and second ports 124, 126 may each include a needle-free connector.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A catheter assembly comprising:
a catheter adapter comprising:
a distal end;
a proximal end;
a lumen extending between the distal end and the proximal end; and
a side port arranged between the distal end and the proximal end, the side port in fluid communication with the lumen;
a catheter secured to the distal end of the catheter adapter and extending distally from the catheter adapter;
a fluid conduit having a proximal end coupled to the side port and a distal end, the fluid conduit in fluid communication with the side port;
an elongated arm configured to extend distally away from the catheter adapter; and
a stabilization platform spaced from the catheter adapter and positioned along the fluid conduit.

2. The catheter assembly of claim 1, further comprising a second elongated arm coupled to the catheter adapter and configured to extend distally away from the catheter adapter, wherein the elongated arms are arranged on opposite sides of the catheter adapter.

3. The catheter assembly of claim 2, wherein the elongated arms are configured to intersect one another at a location distal to the distal end of the catheter adapter.

4. The catheter assembly of claim 3, wherein the stabilization platform is coupled to at least one of the elongated arms.

5. The catheter assembly of claim 4, wherein the stabilization platform is arranged at an intersection of the elongated arms.

6. The catheter assembly of claim 3, wherein the stabilization platform is spaced from the elongated arms.

7. The catheter assembly of claim 1, wherein the fluid conduit extends distally relative to the catheter.

8. The catheter assembly of claim 1, further comprising an access connector arranged at the distal end of the fluid conduit.

9. The catheter assembly of claim 8, wherein the access connector comprises a needle-free connector.

10. The catheter assembly of claim 1, wherein the stabilization platform comprises an attachment member configured to secure the stabilization platform to a skin surface of a patient.

11. The catheter assembly of claim 10, wherein the attachment member comprises an adhesive pad.

12. The catheter assembly of claim 1, further comprising a clamp positioned along the fluid conduit, wherein the stabilization platform is positioned between the clamp and the distal end of the fluid conduit.

13. The catheter assembly of claim 1, further comprising a clamp positioned along the fluid conduit, wherein the stabilization platform is positioned between the clamp and the side port of the catheter adapter.

14. The catheter assembly of claim 1, wherein the stabilization platform is moveable along the fluid conduit.

15. The catheter assembly of claim 1, further comprising a force controlled release connector coupled to the distal end of the fluid conduit, wherein at least a portion of the force controlled release connector is configured to separate from fluid conduit when a predetermined separation force is applied to the force controlled release connector.

16. The catheter assembly of claim 15, wherein the force controlled release connector comprises a disconnect connector and a release feature, the release feature is engaged with the disconnect connector and configured to be disengaged from the disconnect connector when a predetermined separation force is applied to the disconnect connector.

17. The catheter assembly of claim 16, wherein the disconnect connector comprises a valve member.

18. The catheter assembly of claim 16, wherein the disconnect connector comprises a first line extending from the disconnect connector and a second line extending from the disconnect connector, and wherein the first line comprises a first port and the second line comprises a second port.

19. The catheter assembly of claim 18, wherein the first and second ports each comprises a needle-free connector.
